(19) United States
(12) Patent Application Publication
(10) Pub. No.: US 2004/0247558 A1
(43) Pub. Date: Dec. 9, 2004

(54) Shaving liquid for a shaver

(76) Inventors: Petra Allef, Essen (DE); Dietmar Korn, Eschborn (DE); Gerhard Finger, Schmitten (DE)

Correspondence Address: FISH & RICHARDSON PC, 225 FRANKLIN ST, BOSTON, MA, 02110, US

(21) Appl. No.: 10/850,782

(22) Filed: May 21, 2004

Related U.S. Application Data
(63) Continuation of application No. PCT/EP02/12743, filed on Nov. 14, 2002.

(30) Foreign Application Priority Data
Nov. 21, 2001 (DE) 10156574.7

(51) Int. Cl. A61K 7/15

(52) U.S. Cl. 424/73

(57) ABSTRACT

A shaving liquid in a hair removal device is described. The shaving liquid includes one or more alcohols, one or more emollients, one or more emulsifiers, and/or other additives and active substances.

### CROSS-REFERENCE TO RELATED APPLICATIONS

This is a continuation of PCT application serial no. PCT/EP02/12743, filed Nov. 14, 2002, which claims priority to German Patent Application No. 101 56 574.7, filed on Nov. 21, 2001, which is incorporated herein by reference in its entirety.

### TECHNICAL FIELD

The present invention relates to a shaving liquid in a hair removal device, for example in an electric dry shaver.

### BACKGROUND

Conventional electric dry shavers may be uncomfortable in shaving and cause skin irritations with sensitive persons as well as persons with a normal skin that is moist, for example, due to the climate or physical efforts. In some instances, the cause of skin irritations is an increased sliding friction of the shaver on moist skin. For example, a dynamic sliding friction coefficient ranging between 0.3 to 0.4 with dry skin can rise up to about 1.5 when the skin is moist.

One method used to address this problem is the use of pre shave products. The pre shave products generally dry the skin and thus cause a lower friction on the skin. Another benefit of some pre shave products includes causing erection of the users hair, which can allow the shaver to better grip the hair. However, it is generally a disadvantage that the use of a pre shave product requires up to two additional treatment steps. These additional treatment steps result because pre shave is generally applied prior to shaving, remains on the skin for roughly two minutes to achieve the desired drying effect and, depending on the pre-shave's formulation, must be washed off again after shaving.

U.S. Pat. No. 5,914,103 describes a pre shave product that is generally applied before shaving and rubbed into the skin after shaving. One purported advantage disclosed in this patent is the lack of a need to wash off the pre shave product after shaving. However, this pre shave product contains much water, which during application and during shaving can increase the friction between the shaving elements of the shaver and the skin.

The after shave disclosed in DE 69 508 764 T2 also includes a significant amount of water. Although a combination of grease and lactic acid ensures that absorbed moisture is retained in the skin, the result is generally that the friction in the contact area between the head of the shaver and the skin is increased.

A shaving liquid or gel for an electric shaver is described in WO 98/47474. The gel or the liquid is generally applied during shaving and has the effect of a shaving cream. A potential disadvantage of such a liquid or gel includes the fact that when the liquid becomes dry, the sliding friction can rise to an unfavorable extent. Moreover, the liquid or gel does not generally have a hair-erecting effect. Additionally, the formulation requires removal, for example by washing after shaving, as it can leave a sticky film on the skin. Because the liquid requires removal from the skin after shaving, for example by washing it off with water, the user is generally not able to shave at any place desired. Moreover, the ingredients may only have a low skin care effect because the formulation is removed after shaving.

After shaves generally suffer from the shortcoming that they represent an additional step of treatment. Additionally, because they are commonly applied after shaving, they mitigate skin irritations only after shaving, as opposed to actually preventing skin irritations.

Published application DE 199 07 025 A1 describes a hair removal device, such as a dry shaver with a liquid transport system. The liquids to be stored and output refer to auxiliary means for shaving such as a pre shave or an after shave and/or any lubricant for improving the sliding movements of a razor blade on the skin.

### SUMMARY

An object of the invention is to provide a shaving liquid in a hair removal device that overcomes some of the disadvantages of the state of the art described above. Additional objectives of the shaving liquid include providing a pleasant skin feel during shaving, reducing the friction between the shaver and the skin, and reducing a stick or slip effect of moist skin.

According to one aspect of the invention, one or more of these objects can be addressed by shaving with a hair removal device such as an electric dry shaver that includes a shaving liquid therein. The shaving liquid is generally substantially anhydrous and can include one or more alcohols, one or more emollients, one or more emulsifiers and/or one or more surface active agents. Substantially anhydrous as defined herein refers to compositions including less than about 1%, for example, less than about 0.5% water. Preferred compositions have no water.

In one embodiment, the invention includes a shaving liquid for use in a hair removal device. The shaving liquid includes an alcohol, an emollient that is not a polyhydric alcohol derivative, and an emulsifier, an active substance and/or an additive. The shaving liquid is substantially anhydrous and is substantially free of silicone.

In another embodiment, the invention includes a razor including a shaving liquid that is released during shaving. The shaving liquid includes an alcohol; an emollient; and a component selected from the group consisting of an emulsifier, an active substance and an additive, wherein the shaving liquid is substantially anhydrous

In another embodiment, the invention includes a method of shaving. The method includes shaving with a razor that during shaving releases a liquid including an alcohol; an emollient; and a component selected from the group consisting of an emulsifier, an active substance and an additive. In some instances, the liquid is substantially anhydrous.

The shaving liquid can be applied during shaving as described in U.S. Pat. No. 6,594,905, which is incorporated herein by reference in its entirety. In this arrangement, the formulation of the shaving liquid can be chosen so that it does not affect the plastic components of the hair removal device or the electric dry shaver. For example, the shaving liquid can be substantially free of silicone. Alternatively, the material of the shaver can be conformed to requirements of the shaving liquid.

The phrase “substantially free of silicone” as used herein refers to a shaving liquid that contains less than about 1% by weight of silicone, for example, less than about 0.5% by weight. In preferred embodiments, the shaving liquid contains no silicone.

An after shave lotion is known from DE 28 05 130 A, which includes polymers or a polymeric salt to effect lubrication. However, this additive can be disadvantageous in that it stays as a film on the skin and washing it off is required to avoid an unpleasant skin feel. Accordingly, preferred embodiments of the shaving liquid of the invention are substantially free from polymers. Substantially free from polymers, as used herein, means that the shaving liquid contains less than about 1% of a polymer, for example, less than about 0.5%, and preferably the shaving liquid contains no polymers.

As the shaving liquid is applied to the skin of a user, preferred embodiments are substantially free of toxins, for example diethylphthalate. As used herein, the phrase substantially free of diethylphalate means that the shaving liquid contains less than about 1% of a diethylphthalate, for example, less than about 0.5%, and preferably the shaving liquid contains no diethylphthalate.

In some embodiments, an advantage of the shaving liquid, for example the shaving liquid contained in a hair removal device, is that it can combine a pre shave with an after shave. In another embodiment, the shaving liquid, as applied during shaving, allows the friction of the shaver on the skin to be maintained at a given level or reduced to a lower level. In some embodiments, the shaving liquid does not require rubbing into the skin as it can soak in automatically. In other embodiments, the shaving liquid does not require removal, for example washing off after shaving, because the shaving liquid does not produce a thick sticky layer on the skin. In still other embodiments, the shaving liquid formulation can be selected in so that it is unnecessary to wait for two minutes before shaving, as is required with some commercial pre shaves.

The details of one or more embodiments of the invention are set forth in the description below. Other features, objects, and advantages of the invention will be apparent from the description and from the claims.

### DETAILED DESCRIPTION

In one aspect, the invention relates to the application of shaving liquid during dry shaving. For example, the shaving liquid is applied directly from the hair removal device onto the skin during shaving.

In general, the shaving liquid includes an alcohol, for example, at least one alcohol. Any alcohols generally used in shaving products may be used, however, ethanol is generally preferred.

Substances known, for example in cosmetics, may be used as emollients and emulsifiers. Examples of emollients and emulsifiers include polyethyleneglycol and polypropyleneglycol derivatives. In general, the emollients and emulsifiers act as lubricants.

In certain embodiments, the effects of conventional pre and after shaves are combined in the shaving liquid, which is disposed in a hair removal device. In this instance, it is not necessary to apply an after shave, thus allowing the user to avoid an additional treatment step. The fact that the liquid is applied during shaving and subsequent treatment of the skin is unnecessary can improve the flexibility of shaving, for example, allowing shaving to occur at any time and in any place desired.

In some embodiments, the shaving liquid includes a high percent alcohol, for example at least 70% or at least 85%. In these instances, due at least in part to its alcohol content, the shaving liquid extracts moisture from the skin and thus achieves a significantly reduced sliding friction of the hair removal device on the skin. Additionally, the high percent of alcohol, for example percent by weight, can induce the hair to stand up from the skin. This condition allows the hair removal device a better grip of the hair, thus helping to obtain a closer shave. In contrast to some conventional pre shaves, however, these two effects are achieved by arranging the shaving liquid in a hair removal device during shaving. Another advantage of a high percent alcohol formulation includes prevention of an increase in sliding friction of the shaver on the skin when the shaving liquid becomes dry on the skin.

In instances where the skin is dried by the shaving liquid, improved sliding of the hair removal device on moist or sweaty skin is generally achieved. Accordingly, the shaving can become more gentle and more pleasant. The reduction of the sliding friction can also cause a significant mitigation of skin irritation during shaving.

Where the shaving liquid does not become sticky during and after shaving, there is no need to wash it off after shaving. Accordingly, any active ingredients in the shaving liquid remain on the skin and thus may be able to provide additional skin care. Moreover, the range of application of the hair removal device can also be considerably extended.

In a preferred embodiment, the emulsifier is non-ionic and includes at most one OH-group. In a still more preferred embodiment, the emulsifier belongs to the group of alpha hydroxycarbon acid esters or polyethyleneglycol derivatives or fatty acid esters. Examples of such compounds include alkyl lactate, oleth-5 and isopropylmyristate. In some instances, the lactate is directed to minimizing the friction between the skin and the shaving elements of the shaver. In instances where the hair removal devices tends to stick during the drying phase, for example experiences the stick/slip effect, the shaving liquid can also include at least one vitamin as an additive, for example, Vitamin E Acetate. The additive can provide very good sliding properties both in the moist and the drying condition. Moreover, the additive, for example a vitamin such as Vitamin E, can protect the lactate against ageing.

In some embodiments, a function of the lactate includes keeping the various other additives soluble in the alcohol of the shaving liquid.

In instances where liposome is used as an emollient, it is possible to clean the shaver or parts of the shaver using water, thereby enhancing the user friendliness of the shaving liquid.

In some instances, the shaving liquid may include anti-inflammatory and/or non-irritating substances. In these instances, any skin irritation that is already diminished due to the reduced sliding friction of the shaving liquid will be further mitigated. This effect will occur during shaving, which is distinct from the timing achieved during use of a conventional after shave.

In another embodiment, the shaving liquid can include one or more antioxidants, which can provide stability for the shaving liquid ingredients and can also provide additional skin care.

In some embodiments, to obviate the need for a separate after shave, the shaving liquid can also include a perfume or a fragrance.

In some embodiments, the shaving liquid can include antiseptics and/or fungicides. The inclusion of antiseptics or fungicides can increase the disinfecting effect of the shaving liquid that is already provided by the alcohol. Using antiseptics or fungicides can also augment the preservability of the formulation of the shaving liquid, which is disposed in the hair removal device.

In another embodiment, the shaving liquid can include menthol and one or more menthol derivatives. A cooling effect on the skin can be achieved by these additives, which can make shaving more refreshing and more pleasant.

In preferred embodiments, the shaving liquid includes at least 70%, for example, 70% by weight or 70% by volume, of at least one alcohol. It is especially preferred that the shaving liquid includes at least 85%, for example 85% by weigh or 85% by volume, of at least one alcohol.

In another embodiment, the shaving liquid can include skin care substances such as oils of natural or synthetic origin, lipid, liposome, plant extracts or vitamins. Additional skin care substances can serve to provide smoother skin and counteract skin ageing. More delicate skin care substances can be employed when using microencapsulated active substances.

The application of further additives such as preservatives, coloring substances or other active substances used in cosmetics is also envisioned.

### EXAMPLES

### Example 1

### Formulation of Shaving Liquid

| | |
|---|---|
| 92.9% | Ethanol |
| 5.0% | Alkyl lactate |
| 1.0% | Alpha-bisabolol |
| 0.5% | Vitamin E Acetate |
| 0.3% | Liposome |
| 0.3% | Perfume |
| 100.00% | |

### Example 2

### Formulation of Shaving Liquid

| | |
|---|---|
| 91.5% | Ethanol |
| 5.0% | Alkyl lactate |
| 1.0% | Avocado oil |
| 1.0% | Vitamin E Acetate |
| 1.0% | Alpha-bisabolol |
| 0.5% | Perfume |
| 100.00% | |

### Example 3

### Formulation of Shaving Liquid

| | |
|---|---|
| 90.0% | Ethanol |
| 5.0% | Oleth-5 |
| 3.0% | Avocado oil |
| 1.0% | Vitamin E Acetate |
| 1.0% | Alpha-bisabolol |
| 100.00% | |

### Example 4

### Formulation of Shaving Liquid

| | |
|---|---|
| 94.9% | Ethanol |
| 5.0% | Isopropylmyristate |
| 0.1% | Menthol |
| 100.00% | |

Using the shaving liquid according to any of the four examples described above in a hair removal device can reduce the dynamic coefficient of sliding friction on moist skin from 1.5 to values of between about 0.3-0.4, which represent a range that corresponds to the friction on dry skin. The coefficient of sliding friction generally rises only insignificantly or not at all when the liquid is drying.

A number of embodiments of the invention have been described. Nevertheless, it will be understood that various modifications may be made without departing from the spirit and scope of the invention.

What is claimed is:

1. A shaving liquid for use in a hair removal device, comprising
an alcohol;
an emollient, wherein the emollient is not a polyhydric alcohol derivative; and
a component selected from the group consisting of an emulsifier, an active substance and an additive;
wherein the shaving liquid is substantially anhydrous and is substantially free of silicone.

2. The shaving liquid of claim 1, wherein the emulsifier is non-ionic and includes at most one OH-Group.

3. The shaving liquid of claim 2, wherein the emulsifier is an alpha hydroxycarbon acid ester or a polyethyleneglycol derivative or fatty acid ester.

4. The shaving liquid of claim 3, wherein the emulsifier is alkyl lactate, oleth-5 or isopropylmyristate.

5. The shaving liquid of claim 1, wherein the shaving liquid comprises anti-inflammatory or a non-irritating substance.

6. The shaving liquid of claim 1, wherein the shaving liquid comprises an antioxidant.

7. The shaving liquid of claim 1, wherein the shaving liquid comprises a perfume or fragrance.

8. The shaving liquid of claim 1 wherein the shaving liquid comprises an antiseptic or fungicide.

9. The shaving liquid of claim 1, wherein the shaving liquid comprises menthol or one or more menthol derivatives.

10. The shaving liquid of claim 1, wherein the shaving liquid comprises at least 70% alcohol.

11. The shaving liquid of claim 1, wherein the shaving liquid comprises at least 85% alcohol.

12. The shaving liquid of claim 1, wherein the shaving liquid is substantially free of diethylphthalate.

13. The shaving liquid of claim 1, wherein the shaving liquid is substantially free of polymers.

14. A razor comprising a shaving liquid that is released during shaving, the shaving liquid comprising an alcohol; an emollient; and a component selected from the group consisting of an emulsifier, an active substance and an additive, wherein the shaving liquid is substantially anhydrous.

15. The razor of claim 14, wherein the shaving liquid is substantially free of diethylphthalate.

16. The razor of claim 14, wherein the shaving liquid is substantially free of silicone.

17. The razor of claim 14, wherein the emollient is not a polyhydric alcohol derivative.

18. The razor of claim 14, wherein the razor is an electric dry shaver.

19. The razor of claim 14, wherein the shaving liquid is substantially free of polymers.

20. A method of shaving, comprising shaving with a razor that during shaving releases a liquid comprising an alcohol; an emollient; and a component selected from the group consisting of an emulsifier, an active substance and an additive.

21. The method of claim 20, wherein the shaving liquid is substantially free of diethylphthalate.

22. The method of claim 20, wherein the shaving liquid is substantially free of Silicone.

23. The method of claim 20, wherein the emollient is not a polyhydric alcohol derivative.

24. The method of claim 20, wherein the shaving liquid is substantially free of polymers.

25. The method of claim 20, wherein the razor is an electric dry shaver.
